(19) United States
(12) Plant Patent Application Publication
(10) Pub. No.: US 2011/0239344 P1
(43) Pub. Date: Sep. 29, 2011

(54) Calibrachoa plant named 'KLECA09210'

(50) ;

(75) Inventors: Nils Klemm, Stuttgart (DE); Anita Stoever, Ostfildern (DE)

(73) Assignee: KLEMM+SOHN GMBH & CO. KG, Stuttgart (DE)

(21) Appl. No.: 12/661,881

(22) Filed: Mar. 25, 2010

(51) Int. Cl. A01H 5/00

(52) U.S. Cl. PLT/413

(57) ABSTRACT

A new Calibrachoa plant named ‘KLECA09210’ particularly distinguished by its single, purple flowers, and a semi-trailing form and growth habit, is disclosed.

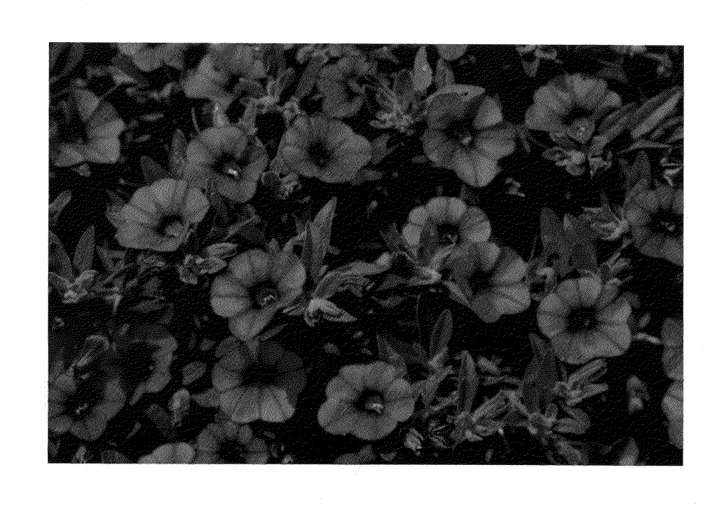

### GENUS AND SPECIES

Calibrachoa hybrid

### VARIETY DENOMINATION

‘KLECA09210’

### BACKGROUND OF THE NEW PLANT

The present invention comprises a new and distinct variety of Calibrachoa, botanically known as Calibrachoa hybrid and hereinafter referred to by the variety name ‘KLECA09210’. The new variety originated from an open pollination made in June 2006 in Stuttgart, Germany. The female parent was the proprietary Calibrachoa plant ‘CA060011’ (unpatented), while the male parent was an un-named individual Calibrachoa plant.

The new variety was first propagated via vegetative cuttings in May 2007 in Stuttgart, Germany and has been asexually reproduced by vegetative cuttings and in-vitro propagation in Stuttgart, Germany for more than 30 generations. The present invention has been found to retain its distinctive characteristics through successive asexual propagations.

‘KLECA09210’ has not been made publicly available or sold more than one year prior to the filing date of this application.

### SUMMARY OF THE INVENTION

The following are the most outstanding and distinguishing characteristics of this new variety when grown under normal horticultural practices in Stuttgart, Germany.
1. Single, purple flowers; and
2. A semi-trailing form and growth habit.

### DESCRIPTION OF THE PHOTOGRAPH

This new Calibrachoa plant is illustrated by the accompanying photograph which shows flowers and foliage of the plant. The colors shown are as true as can be reasonably obtained by conventional photographic procedures. The photograph was taken in May 2009 of a 5-month-old Calibrachoa plant, grown in a glass greenhouse in Stuttgart, Germany.

### DESCRIPTION OF THE NEW VARIETY

The following detailed descriptions set forth the distinctive characteristics of ‘KLECA09210’. The data which defines these characteristics were collected from asexual reproductions carried out in Stuttgart, Germany. The plant history was taken in May 2009 on 5-month-old plants which were planted as rooted cuttings in a 10 centimeter pot and then grown in a glass greenhouse. The plants had no terminal pinches after potting. Color readings were taken under natural light. Color references are primarily to the R.H.S. Colour Chart of The Royal Horticultural Society of London (R.H.S.), Fifth Edition (2007).

### DETAILED BOTANICAL DESCRIPTION OF THE NEW PLANT

Classification:
   Family.—Solanaceae.
   Botanical.—Calibrachoa sp.
   Denomination.—‘KLECA09210’.
   Common.—Calibrachoa Hybrida.
Parentage:
   Female.—The proprietary Calibrachoa plant ‘CA060011’ (unpatented).
   Male.—An un-named individual Calibrachoa plant.
Growth:
   Form.—Semi-trailing.
   Habit.—Semi-trailing.
   Height (measured from the top of the soil).—6.0 cm.
   Width (horizontal plant diameter).—17.0 cm.
   Time to produce a finished flowering plant.—9 weeks.
   Outdoor plant performance.—Continuously flowering, healthy, good rain performance.
   Time to initiate and develop roots.—16 days.
   Root description.—Moderate density, moderate branching, white roots.
Stems:
   Length of basal branches.—9.0 cm.
   Internode length.—2.0 cm.
   Diameter of branches (from midpoint).—5.0 cm to 6.0 cm.
   Color.—RHS 145B.
   Texture.—Smooth.
   Anthocyanin.—No.
   Pedicel.—Color: RHS 144B Length: 1.3 cm Diameter: 0.2 cm Texture: Smooth.
Leaves:
   Arrangement.—Alternate.
   Size.—Length: 2.5 cm to 3.5 cm Width: 0.9 cm.
   Shape.—Lanceolate to elliptic.
   Apex.—Broad acute.
   Base.—Attenuate.
   Margin.—Entire.
   Color (
   immature and mature leaves).—Upper surface: RHS N138B Lower surface: RHS N138C.
   Texture (
   both upper and lower surfaces).—Smooth.
   Venation pattern.—Pinnate.
   Venation color (both upper and lower surfaces).—RHS 144A.
   Petioles.—Absent.
Flower bud:
   Shape.—Irregular to oblong.
   Size.—Length: 1.5 cm Diameter: 0.5 cm.
   Color at tight bud.—RHS N144A.
Inflorescence:
   Blooming habit.—Continuous flowering during summertime.
   Inflorescence type.—Single flower, determinate inflorescence.
   Floret type.—Funnel-shaped, single flower.
   Lastingness of individual blooms on the plant.—5 days.
   Fragrance.—Absent.
Flowers:
   Immature flower.—Diameter: Horizontal: 2.3 cm Vertical: 2.0 cm Color: Upper surface: RHS 60C Lower surface: RHS 64A.
   Mature flower.—Diameter: Horizontal: 2.8 cm Vertical: 2.0 cm. Color: Upper surface: RHS 60C Lower surface: RHS 64A.
   Corolla tube.—Color inside: RHS 6A Color outside: RHS 1C Tube length: 1.0 cm Texture (outside): Moderate Texture (inside): Smooth.
   Petals.—Apex: Obverse Base: Open, fused into the tube Margin: Entire Waviness of petals: Weak Lobation: Weak Texture (both upper and lower surfaces): Smooth.
   Sepals.—Calyx (composition): Rosette, actinomorphic Shape: Lanceolate to elliptic Apex: Acute Base: Open, tube fused into the base Margin: Entire Length: 1.3 cm Width: 0.3 cm Color: Upper surface: RHS 138A Lower surface: RHS 138B Texture (both upper and lower surfaces): Smooth.
Reproductive organs:
   Stamens.—Number: 5 Filament color: RHS 154A Filament length: 0.7 cm Filament diameter: 0.1 cm Pollen color: RHS 11A Pollen quantity: Sparse.
   Pistils.—Number: 1 Length: 0.3 cm Diameter: 0.2 cm Stigma color: RHS 149A.
   Style color.—RHS 149A.
Fruit and seed set: No fruit or seed set observed.
Disease and insect resistance: No particular resistance or susceptibility has been observed.
Comparison with parental and commercial varieties: ‘KLECA09210’ differs from the female parent Calibrachoa plant ‘CA060011’ (unpatented) in that ‘KLECA09210’ has purple flowers, while ‘CA060011’ has dark blue flowers. ‘KLECA09210’ differs from the commercial variety ‘MiniFamous Burgundy’ (unpatented) in that ‘KLECA09210’ has more branching and a larger flower size than ‘MiniFamous Burgundy’.

We claim:

1. A new and distinct variety of Calibrachoa plant named ‘KLECA09210’ as shown and described herein.
